(19) United States
(12) Patent Application Publication
(10) Pub. No.: US 2015/0223745 A1
(43) Pub. Date: Aug. 13, 2015

(54) BLADDER-PRESSURE MEASUREMENT CAPSULE

(71) Applicant: Universitaet zu Köln, Köln (DE)

(72) Inventors: Sebastian Wille, Koeln-Weiden (DE); Jan Mehner, Neukirchen (DE); Dirk Tenholte, Chemnitz (DE)

(21) Appl. No.: 14/424,062

(22) PCT Filed: Aug. 23, 2013
(86) PCT No.: PCT/EP2013/002546

§ 371 Date: Apr. 10, 2015

(30) Foreign Application Priority Data
Aug. 27, 2012 (DE) 10 2012 016 798.9

(51) Int. Cl. A61B 5/20; B29C 70/70

(52) U.S. Cl. CPC A61B 5/205; B29C 70/70; A61B 2562/12; B29K 2021/003

(57) ABSTRACT

The invention relates to a bladder measurement capsule, which can be introduced into the bladder of a living being, in particular a human, and which has a pressure sensor and measurement electronics in a housing by means of which pressure measurement values provided by the pressure sensor can be detected and saved and which has a shape deviating from a linear longitudinal extension, in particular the shape of a ring torus sector, and is flexible against a resetting force into a linear longitudinally extended shape. The invention further relates to a method for producing a bladder pressure measurement capsule, wherein a flexible circuit board (2) carrying the internal components, in particular the electronic components, is inserted into a mold cavity which defines a shape of the bladder pressure measurement capsule deviating from a linear longitudinal extension, in particular the shape of a ring torus sector, and the mold cavity is filled with an elastomer casting compound (1) which cures without tension, in particular silicone.

The invention relates to a bladder-pressure measurement capsule that can be introduced into the bladder of a living being, in particular a human, and that has a housing comprising a pressure sensor and measurement electronics by means of which pressure measurements provided by the pressure sensor can be detected and stored. The invention further relates to a method of making a bladder-pressure measurement capsule.

Bladder-pressure measurement capsules are principally known in the prior art and can be used, for example, in medical diagnostics for examining patients suffering from a so-called overactive bladder. According to the known prior art, bladder-pressure measurement capsules of this kind has a straight longitudinally extending cylindrical housing that usually has part-spherical or rounded ends and thus can be inserted through the urethra into the bladder of a human by a cystoscope.

For recording measurements of the bladder pressure, such measurement capsules further include a pressure sensor as well as usually battery-operated electronics so as to detect pressure measurements over time and to store them in an internal memory for later reading and subsequent evaluating of the recorded measurements for diagnostic purposes.

It is regarded to be a problem of these bladder-pressure measurement capsules that the positive property, namely to be able to introduce such a bladder-pressure measurement capsule through the urethra into the bladder by a cystoscope, at the same time poses the high risk that during urination such a bladder-pressure measurement capsule finds its way from the bladder back into the urethra and either closes the urethra or is flushed out.

It is therefore an object of the invention to provide a bladder-pressure measurement capsule of the aforementioned generic kind that, despite being easy to insert with a cystoscope, involves at the same a significantly reduced risk of unintentionally getting into the urethra during urination.

This object is achieved according to the invention in that the bladder-pressure measurement capsule has a elongated nonstraight shape and can be bent against a resetting force into an elongated and straight shape.

The essential central idea of the invention therefore is to provide such a bladder-pressure measurement capsule that can be bent elastically from its basic shape, thus the shape that is present if no forces whatsoever are exerted on the capsule, into a straight, that is, straight longitudinally extending shape and, like the bladder-pressure measurement capsules known heretofore in the prior art, thus can be inserted through the urethra into the bladder by a cystoscope, and that after releasing from the cystoscope bends back into its basic shape as a result of the acting resetting force, that is, into a shape that deviates from the elongated and straight shape.

By the reshaping of the bladder-pressure measurement capsule according to the invention after releasing from the cystoscope into its shape deviating from the elongated and straight shape, the capsule cannot be easily flushed out of the bladder because even if an end of the bladder-pressure measurement capsule should be able to find its way into the inlet of the urethra, the rest of the bladder-pressure measurement capsule cannot follow due to the shape deviating from a elongated and straight shape. It is basically possible to achieve this effect according to the invention by any elongated nonstraight shape and that is assumed by the bladder-pressure measurement capsule if no external forces whatsoever are exerted on it.

In a preferred embodiment of the invention the shape deviating from the elongated and straight shape is such that the bladder-pressure measurement capsule forms an arc, or, as described in geometrical terms, has the shape of a toroidal segment.

Such an arc or toroidal segment can be formed to be substantially circular in its cross-section perpendicular to its extent so that by bending the bladder-pressure measurement capsule having this shape into a elongated and straight shape, a cylindrical shape, like the bladder-pressure measurement capsules known from the prior art, is obtained, including the advantages of easy insertion by a cystoscope.

With this shape of a toroidal segment, at least one end, optionally both longitudinal ends of the bladder-pressure measurement capsule, can be rounded or part spherical, in particular to be hemispherical, to avoid injuries caused by the ends of such a capsule.

Even with the cross-sectional shape perpendicular to the longitudinal extent not being exactly circular, a basically bent, in particular toroidal-segmental shape can be used, with which shape at least the imaginary center line running through the bladder-pressure measurement capsule has a bend or curvature, in particular in only one plane, and this bend thus defines an arc or a C-shape or a section of the periphery of a circle.

In addition to the above-described preferred bent shape, of which a bend is substantially present in only one plane, the bend defines a wave. Likewise, a bend of the bladder-pressure measurement capsule or of the housing of the measurement capsule in which the components are received, is bent in more than one plane or dimension. For example, a helical formation of such a bladder-pressure measurement capsule is also conceivable.

In a preferred refinement of the bladder-pressure measurement capsule according to the invention the housing surrounding the inner components such as, for example, the measurement electronics and the pressure sensor, is formed from a flexible material that allows bending from the basic shape deviating from the elongated and straight shape into a straight shape, that is, elongated and straight shape. For example, a flexible casting compound, for example an elastomer, can be selected for such a flexible material that is cast around the inner components. In an exemplary application, silicone can be used that has high biocompatibility.

In another embodiment at least the measurement electronics, in particular further internal components of the bladder-pressure measurement capsule, are mounted on a flexible circuit board. This has the advantage that when bending the bladder-pressure measurement capsule out of its basic shape into the elongated and straight shape, the internal components of the bladder-pressure measurement capsule can also follow this bending without any problems and without getting damaged.

Thus, for example, a flexible circuit board together with the housing, in the shape deviating from the elongated and straight shape as well as in the elongated and straight shape generated by the bending, can each have center lines running parallel. This is in particular to be understood such that geometrical center lines or center-of-gravity lines of the housing or the flexible circuit board run in the same direction, are in particular parallel to one another (at least in the elongated and straight shape) or even coincide exactly with one another.

Unless one flexible circuit board is used to carry all the electronic components, one can also use a plurality of rigid circuit board sections that are in particular aligned in a row and that each carry in part electronic or other internal components of the bladder-pressure measurement capsule and are flexibly, that is, bendably connected to one another either by cables or by flexible circuit board sections.

As described above, in the relaxed state, that is, when no external forces act on the bladder-pressure measurement capsule, the bladder-pressure measurement capsule has a shape that deviates from an elongated straight shape, has in particular a bend that lies in one plane. For the effect according to the invention, namely that after inserting the bladder-pressure measurement capsule into the bladder, the bladder-pressure measurement capsule automatically reverts from the straight elongated shape into its basic shape deviating therefrom, a resetting force is required that acts until the bladder-pressure measurement capsule has returned to its basic shape after insertion into the bladder.

In an embodiment according to the invention, the resetting force is at least partially generated, optionally even completely generated by elastic return forces in the material of the casting compound of the housing. Thus, for example, during manufacture of a bladder-pressure measurement capsule according to the invention, the housing with internal components enclosed by the housing is cast stress-free into the desired nonstraight shape by a casting compound that, due to the elasticity of the material, subsequently generates resetting forces only when bent out of the basic shape.

Thus, for example, a resetting force is even applied completely by the casting compound of the housing so that no further elements providing the resetting force are required in a bladder-pressure measurement capsule according to the invention.

In another embodiment, which can also be combined with the previous one, the resetting force is generated at least partly, optionally completely, by elastic resetting forces that are created in the material of the flexible circuit board when the circuit board is bent.

Accordingly, one can use a flexible circuit board that, in the stress-free state, has a bend or a shape that deviates from a straight elongated shape, and that is assembled in this shape with the internal components, in particular at least with the electronic components of the bladder-pressure measurement capsule. When bending such a flexible circuit board into a straight shape, the circuit board itself therefore will provide return forces so as to revert to the initial position.

Of course, one can also use flexible circuit boards in a bladder-pressure measurement capsule that are completely straight or form a flat plane in the stress-free state. Such circuit boards can be bent out of this plane position without any problems and can be kept in this shape, for example, if a flexible or elastic casting compound is cast around the circuit boards, and the resetting forces, which are usually generated by such flexible circuit boards, are too low to allow the circuit board to return to the planar shape after casting.

In all the above-described embodiments the resetting force is generated at least partially, optionally completely, by at least one spring element, in particular a spring band in the housing, in particular surrounded by the housing. If, for example, resetting forces that optionally are provided by the housing and/or are provided by a circuit board, are not sufficient to define a basic nonstraight shape of the bladder-pressure measurement capsule in the stress-free state, suitable spring elements can be added to the internal functional components in the housing.

Thus, one can connect the above-mentioned at least one spring element such as, for example, a spring band, in a firmly bonded manner to a flexible circuit board that carries the internal components, in particular at least the electronic internal components of the bladder-pressure measurement capsule. For example, a firmly bonded connection can be established by adhesion. Thus, the firmly bonded connection forms a substantially integral component whose shape is defined by the shape of the spring element. Accordingly, the spring element can exhibit a bend in one plane to define the C-shape or form the above-described toroidal segment of the entire bladder-pressure measurement capsule, and also any other shape.

If a firmly bonded connection is not provided between the at least one spring element and a circuit board, such a spring element or optionally a plurality of spring elements together can be enclosed by the elastic casting compound in order to form a bladder-pressure measurement capsule according to the invention.

In particular, to ensure that a bladder-pressure measurement capsule can be removed from the bladder after completion of its measuring tasks, in a further development a grippable formation is mounted at least at one end of the bladder-pressure measurement capsule. Such a grippable formation can be provided to be gripped by a gripping tool such as, for example, forceps that are introduced into the bladder by a cystoscope. By the gripping of the grippable formation, the bladder-pressure measurement capsule can be pulled into end of the cystoscope, and the bladder-pressure measurement capsule, due to its flexibility or bendability, can be automatically pulled into the straight shape and thus can be completely enclosed by the cystoscope.

A method of making a bladder-pressure measurement capsule of the above-described inventive kind can provide, for example, that a particularly flexible circuit board carrying the internal components, in particular at least the electronic components, is inserted into a mold cavity that defines a desired nonstraight shape of the bladder-pressure measurement capsule to be produced, such as, for example, the shape of a toroidal segment, and the mold cavity is subsequently filled with an elastomer casting compound, for example silicone, that cures in a stress-free manner.

Accordingly, by casting this elastomer casting compound, the casting compound forms the outer housing of the bladder-pressure measurement capsule according to the invention, and in the cured stress-free state, namely after removal from the mold cavity, it assumes the inventive nonstraight shape such as, for example a bent shape, in particular the shape of a toroidal segment.

As previously described, if material-created resetting forces during bending of the casting compound of the housing are not sufficient for permanently defining a shape that deviates from the straight elongated shape, additional shape-defining spring elements can be put into the mold cavity and cast in place, or inserted not just loosely, but optionally connected to the circuit board in a firmly bonded manner, for example by adhesive bonding.

As previously described, a component provided as a flexible circuit board can be straight or planar when unstressed and thus is bent when being inserted into the mold cavity, or an already pre-shaped circuit board of the flexible kind is used that, in the stress-free state, corresponds substantially to the shape of the bladder-pressure measurement capsule to be produced.

An embodiment of the invention is described in the figures below.

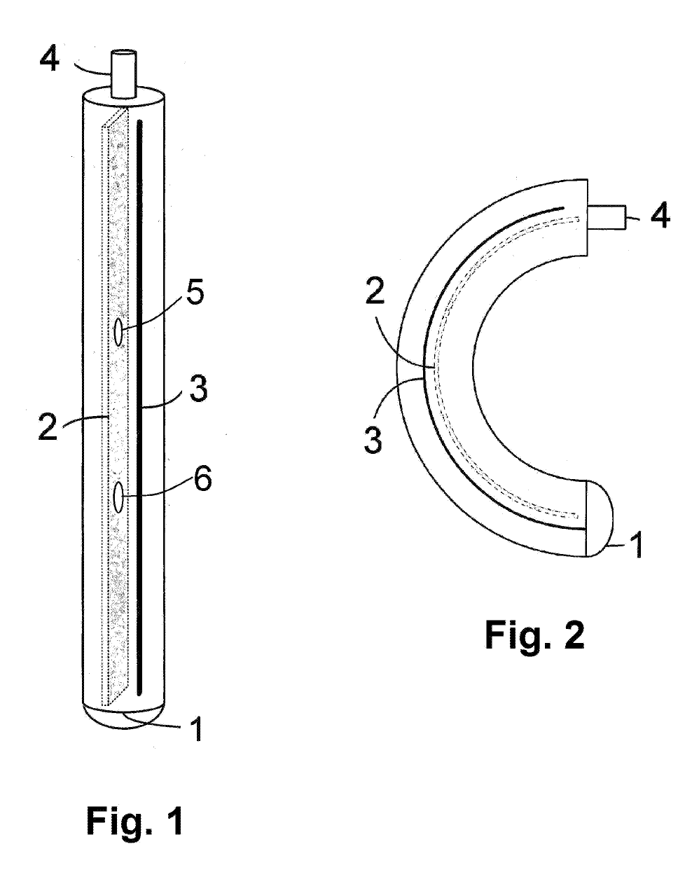

FIG. 1 shows an embodiment of a bladder-pressure measurement capsule according to the invention in a bent state, thus in a state in which such a bladder-pressure measurement capsule is arranged, for example, in unillustrated cystoscope. In such a straight shape that is different from the basic shape, the bladder-pressure measurement capsule is substantially cylindrical with a longitudinal axis that runs vertical in FIG. 1. The bladder-pressure measurement capsule is here made from a casting compound 1 that forms the housing, that is composed for example of silicone, and that surrounds in this embodiment a flexible circuit board 2 with an additional spring element 3 such as, for example, a spring band or wire. At the upper end shown in FIG. 1 there is a grippable formation 4 to enable later removal of the bladder-pressure measurement capsule. The lower end is rounded. The end on which the grippable formation 4 is mounted could also be crowned.

Not shown in FIG. 1, although present, are electronic components that are mounted on the circuit board such as, for example, measurement electronics for detecting pressure measurements that could be detected by a pressure sensor, which likewise is not shown, when the bladder-pressure measurement capsule is inserted into the bladder. For this purpose, measurement electronics on the circuit board 2 further comprise a preferably non-volatile memory in which the bladder-pressure measurements, optionally in connection with further measurements such as, for example temperature measurements, can be stored. A button cell battery that can easily be accommodated in the cross-section of the bladder-pressure measurement capsule can be used as energy source, for example.

Switching on such a bladder-pressure measurement capsule can be carried out in a contactless manner, for example by a magnetically activatable reed switch, for example. After magnetically switching on, the switch can be kept in the closed state by activation by the electronics, for example. The electronics can be configured to periodically record and register pressure measurements, that is, to write them into the memory for later reading. In addition other measurements, such as the temperature in the bladder can be monitored and stored. Here, detecting and storing these other measurements, in particular temperature measurements, can be carried out with a different periodicity, in particular with greater time intervals, than for the pressure measurements.

Also, an internal timer can be provided in the electronics of the measurement capsule so that a timestamp can also be stored along with the measurements. This can help to later correlate the measurements with other chronologically protocolled events, for example urination detected by a pad or by a subjective sensation of micturition. The latter can be indicated by a push-button device actuated by the patient, for example. With each touch of the button, the time when the button was pushed is stored.

FIG. 2 shows the same bladder-pressure measurement capsule according to FIG. 1 in the relaxed state, that is, after the bladder-pressure measurement capsule was released from a cystoscope into the bladder. Resetting forces, which in this embodiment are applied at least predominantly by the at least one spring element 3, deforms the bladder-pressure measurement capsule into a bent body, for example a partly toroidal body such as, for example, a toroidal segment having for example an angular extent around the torus center between 90° and 180°.

This bent shape as shown in FIG. 2 ensures that a relaxed bladder-pressure measurement capsule that freely swims in a bladder does not unintentionally find its way into the bladder outlet during urination and closes the bladder or get flushed out.

By forceps gripping a grippable formation 4, the bent bladder-pressure measurement capsule according to FIG. 2 can be pulled back into a cystoscope, to automatically impart to the bladder-pressure measurement capsule a straight shape according to FIG. 1 and therefore can be removed together with the cystoscope.

In a specific design, the grippable formation can also have a different shape than the shape shown here. For example, it can be formed as a loop or hook or can have a widening, for example undercut head region.

It is to be mentioned that the invention is not limited to the bent, substantially toroidal-segmental shape shown in FIG. 2. Other shapes can also be implemented, and it is merely of essence that in the stress-free basic state, these shapes deviate from straight according to FIG. 1 in order to provide security against being discharged during urination.

1. A bladder measurement capsule that can be introduced into the bladder of a living being, the capsule comprising:
an elastically deformable housing;
a pressure sensor and measurement electronics in the housing operable to detect and store pressure measurements; and
means urging the housing continuously into an elongated nonstraight shape, the housing being elastically bendable against a resetting force into an elongated and straight shape for insertion into the bladder.

2. The bladder-pressure measurement capsule according to claim 1, wherein the housing surrounds the internal components including the measurement electronics and the pressure sensor, and is formed from a flexible material that forms the means and is cast around the inner components.

3. The bladder-pressure measurement capsule according to claim 1, further comprising:
a flexible circuit board on which at least the measurement electronics are mounted.

4. The bladder-pressure measurement capsule according to claim 3, wherein in the elongated nonstraight shape and in the elongated and straight shape generated by bending, the flexible circuit board and the housing each have a common centerline running in the same direction.

5. The bladder-pressure measurement capsule according to any one of the preceding claims, claim 1, wherein the means generating the resetting force is elastic resetting forces in the material of the flexible circuit board and/or in the material of the housing that is cast in a stress-free manner in the desired elongated nonstraight shape.

6. The bladder-pressure measurement capsule according to claim 1, wherein the means generating the resetting force is at least one spring element that is surrounded by the housing.

7. The bladder-pressure measurement capsule according to claim 3, wherein the means is at least one spring element connected to the flexible circuit board by adhesive bonding.

8. The bladder-pressure measurement capsule according to claim 1, further comprising:
a grippable formation on at least one end of two longitudinal ends of the housing.

9. A method of making a bladder-pressure measurement capsule the method comprising the steps lf:
inserting a flexible circuit board carrying electronic components into a mold cavity having a shape that deviates from an elongated and straight shape, and
filling the mold cavity is filled with an elastomer casting compound that cures in a stress-free manner.

10. The method according to claim 9, wherein for generating a resetting force after a deformation from the shape deviating from a elongated and straight shape, the the method further comprises the step or steps of inserting in the mold cavity and imbedding in the casting compound:
a) a flexible circuit board that, in a stress-free state, has an elongated nonstraight shape that in particular corresponds substantially to the shape of the mold cavity, and/or
b) a flexible circuit board that, in the stress-free state, has a elongated and straight shape and is connected in a firmly bonded manner to at least one spring element that has an elongated nonstraight shape that in particular corresponds substantially to the shape of the mold cavity, and/or
c) , in addition to the flexible circuit board, at least one spring element that is not connected to the circuit board and that has an elongated nonstraight shape that in particular corresponds substantially to the shape of the mold cavity.
